(19) United States
(12) Plant Patent Application Publication
(10) Pub. No.: US 2003/0066113 P1
(43) Pub. Date: Apr. 3, 2003

(54) Sedum 'lajos'

(76) Inventor: Brent Horvath, Spring Grove, IL (US)

Correspondence Address: Brent Horvath, 10702 Seaman Rd., Hebron, IL, 60034, US

(21) Appl. No.: 09/898,768

(22) Filed: Oct. 1, 2001

(51) Int. Cl. A01H 5/00

(52) U.S. Cl. PLT/263

(57) ABSTRACT

A new, distinct Sedum characterized by a creamy yellow margin to the foliage.

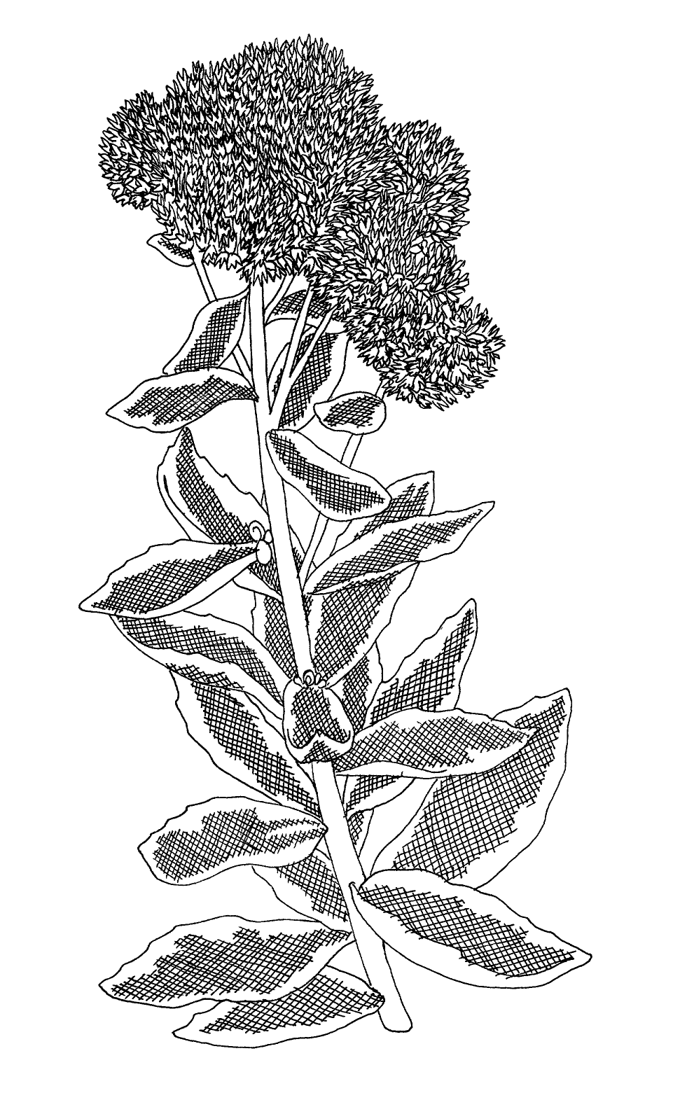

This naturally occuring sport of Sedum ‘Autumn Joy’ (aka ‘Herbstfreude’) has a butter yellow edge to the foliage, compared to the all green leaves of Sedum ‘Autumn Joy’. The original plant was found and purchased from a wholesale perennial nursery in Harvard, Ill. Currently the mother plant is located in a garden at Intrinsic Perennial Gardens, Inc. Hebron, Ill. Asexual, cutting propagation has been the only means of reproduction.

### DESCRIPTION OF PHOTOGRAPHS

1) The variegated sport still on the mother plant.

2) A clump of Sedum ‘Lajos’

3) A close up of the growing tip.

4) Fall flower on Sedum ‘Lajos’

### DETAILED PLANT DESCRIPTION

Foliage:

Type.—Deciduious.

Shape.—Obovate.

Length.—5-8 cm (2-3 inches).

Width.—3-4 cm (1-2 inches).

Thickness.—1-2 mm (⅛ inch).

Margin.—coarsely and irregularly toothed.

Texture.—smooth.

Leaf arrangement.—alternate, crowded except at the base of the stems.

Color.—The variegated edge starts out a creamy yellow and ages to a creamy white. The center of the leaf is green resembling the parent. There is also a shiny appearance giving a metalic or grey color to the foliage.

Enclosed.—one pressed leaf.

Flower:

Petal count.—5.

Flower type.—panicle.

Flower size.—3 mm across, 4 mm tall.

Flower number.—approximately 2000-3000 per mature.

Panicle measuring 15 cm (6 inches).

Blooming habit.—domed, terminal panicle on up to 60 cm (2 ft.) stems.

Bloom period.—september.

Bloom color.—pink flowers age to copper and persist with a rust color in winter.

I claim:

1. A new, distinct Sedum plant as shown and described, characterized by a creamy yellow edge to the foliage. (Unlike the parent plant which has all green foliage.) No other Sedum in commerce has this type of variegation.
